(19) United States
(12) Patent Application Publication
(10) Pub. No.: US 2020/0164034 A1
(43) Pub. Date: May 28, 2020

(54) METHODS FOR IMPROVING SEX-DIMORPHIC RESPONSES TO TARGETED THERAPY IN MELANOMA

(71) Applicant: Board of Regents, The University of Texas System, Austin, TX (US)

(72) Inventors: Jennifer WARGO, Houston, TX (US); Timothy P. HEFFERNAN, Houston, TX (US); Swathi ARUR, Houston, TX (US); Miles C. ANDREWS, Houston, TX (US); Joseph MARSZALEK, Houston, TX (US); Christopher VELLANO, Houston, TX (US)

(21) Appl. No.: 16/663,271

(22) Filed: Oct. 24, 2019

Related U.S. Application Data
(60) Provisional application No. 62/750,083, filed on Oct. 24, 2018.

(51) Int. Cl. A61K 38/17; A61K 31/506; A61K 31/519; C07K 16/28; A61P 35/00

(52) U.S. Cl. CPC A61K 38/1796; A61K 31/506; A61K 31/519; A61K 45/06; C07K 16/2818; A61P 35/00; C07K 16/2827

(57) ABSTRACT

Provided herein are methods for the treatment of BRAF-mutant cancer with targeted therapies including androgen receptor (AR) antagonists, G Protein-Coupled Estrogen Receptor 1 (GPER1) agonists, and/or MYC inhibitors. The therapies may be combined with BRAF and MEK-targeted therapy and/or immune checkpoint blockade.

This application claims the benefit of U.S. Provisional Application No. 62/750,083, filed Oct. 24, 2018, the entirety of which is incorporated herein by reference.

### BACKGROUND

### 1. Field

The present invention relates generally to the fields of medicine and immunology. More particularly, it concerns methods for methods of treating cancer with targeted therapy.

### 2. Description of Related Art

An individual's sex has been long recognized as a key factor affecting cancer incidence, prognosis, and treatment responses. However, the molecular basis for sex disparities in cancer remains poorly understood. A comprehensive analysis of molecular differences between male and female patients was performed in 13 cancer types of The Cancer Genome Atlas and revealed two sex-effect groups associated with distinct incidence and mortality profiles. One group contained a small number of sex-affected genes, whereas the other showed much more extensive sex-biased molecular signatures. Importantly, 53% of clinically actionable genes (60/114) showed sex-biased signatures. The study provided a systematic molecular-level understanding of sex effects in diverse cancers; however, there is an unmet need to develop sex-specific therapeutic strategies in certain cancer types.

### SUMMARY

In certain embodiments, the present disclosure provides methods of treating a an androgen receptor (AR) antagonist to the subject. In some aspects, the subject is male. In particular aspects, the BRAF mutation is V600E. In certain aspects, the subject has undergone surgical resection. In certain aspects, the subject has undergone castration. In some aspects, the cancer is melanoma, such as high-risk resectable melanoma or metastatic melanoma. In some aspects, the AR antagonist is enzalutamide, bicalutamide, lutamide, nilutamide, ketonazole, abiraterone, abiraterone acetate, orteronel, finasteride, dutasteride, bexlosteride, izonsteride, turosteride, episteride, dexamethasone, prednisone, leuprolide, goserelin, triptorelin, histrelin, or estrogen.

In additional aspects, the method further comprises administering a BRAF and MEK-targeted therapy, such as dabrafenib and/or trametinib. The dafrafenib may be administered at a dose of 100-200 mg, such as 150 mg and trametinib may be administered at a dose of 1-5 mg, such as 2 mg.

In further aspects, the method further comprises administering an immune checkpoint blockade (ICB) therapy. In some aspects, the ICB therapy is administered intravenously. The ICB therapy may comprise one or more inhibitors of CTLA-4, PD-1, PD-L1, PD-L2, LAG-3, BTLA, B7H3, B7H4, TIM3, KIR, or A2aR. For example, the ICB therapy comprises an anti-PD1 antibody and/or an anti-CTLA4 antibody. In some aspects, the anti-PD1 antibody is nivolumab, pembrolizumab, pidillizumab, AMP-514, REGN2810, CT-011, BMS 936559, MPDL328OA or AMP-224. In particular aspects, the anti-CTLA-4 antibody is tremelimumab or ipilimumab.

In some aspects, the method further comprises administering at least one additional anti-cancer therapy. In certain aspects, the anti-cancer therapy is chemotherapy, immunotherapy, targeted therapy, surgery, radiotherapy, or biotherapy. In some aspects, the anti-cancer therapy is administered orally, intravenously, intraperitoneally, intratracheally, intratumorally, intramuscularly, endoscopically, intralesionally, percutaneously, subcutaneously, topically, regionally, or by direct injection or perfusion. In some aspects, the AR antagonist and/or at one additional anti-cancer therapy is administered simultaneously. In certain aspects, the AR antagonist is administered prior to the at least one additional anti-cancer therapy.

In another embodiment, there is provided a composition comprising an effective amount of an AR antagonist and a BRAF and MEK-targeted therapy for use in the treatment of BRAF mutant cancer in a subject. In some aspects, the BRAF and MEK-targeted therapy is dabrafenib and trametinib. The dafrafenib may be administered at a dose of 100-200 mg, such as 150 mg and trametinib may be administered at a dose of 1-5 mg, such as 2 mg.

In some aspects, the subject is male. In particular aspects, the BRAF mutation is V600E. In certain aspects, the subject has undergone surgical resection. In some aspects, the cancer is melanoma, such as high-risk resectable melanoma or metastatic melanoma.

In some aspects, the AR antagonist is enzalutamide, bicalutamide, lutamide, nilutamide, ketonazole, abiraterone, abiraterone acetate, orteronel, finasteride, dutasteride, bexlosteride, izonsteride, turosteride, episteride, dexamethasone, prednisone, leuprolide, goserelin, triptorelin, histrelin, or estrogen.

In further aspects, the composition further comprises administering an immune checkpoint blockade (ICB) therapy. The ICB therapy may comprise one or more inhibitors of CTLA-4, PD-1, PD-L1, PD-L2, LAG-3, BTLA, B7H3, B7H4, TIM3, KIR, or A2aR. For example, the ICB therapy comprises an anti-PD1 antibody and/or an anti-CTLA4 antibody. In some aspects, the anti-PD1 antibody is nivolumab, pembrolizumab, pidillizumab, AMP-514, REGN2810, CT-011, BMS 936559, MPDL328OA or AMP-224. In particular aspects, the anti-CTLA-4 antibody is tremelimumab or ipilimumab.

In certain embodiments, the present disclosure provides methods of treating a BRAF-mutant cancer in a subject comprising administering a G Protein-Coupled Estrogen Receptor 1 (GPER1) agonist and/or an inhibitor of MYC to the subject. In some aspects, the subject is male. In particular aspects, the BRAF mutation is V600E. In certain aspects, the subject has undergone surgical resection. In some aspects, the cancer is melanoma, such as high-risk resectable melanoma or metastatic melanoma.

In some aspects, the GPER agonist is G-1, fulvestrant, raloxifene, or tamoxifen. The GPER1 agonist may be administered as an adjuvant therapy. In certain aspects, the inhibitor of MYC is a small molecule antagonist, CRABBP inhibitor, BET inhibitor, CDK7 inhibitor, CDK9 inhibitor, mTOR inhibitor, anti-Myc antibody or antigen-binding fragment thereof, an aptamer, siRNA, shRNA, or microRNA. The BET inhibitor may be JQ1 or GSK525762.

In additional aspects, the method further comprises administering a BRAF and MEK-targeted therapy, such as dabrafenib and/or trametinib. The dafrafenib may be administered at a dose of 100-200 mg, such as 150 mg and trametinib may be administered at a dose of 1-5 mg, such as 2 mg.

In further aspects, the method further comprises administering an immune checkpoint blockade (ICB) therapy. In some aspects, the ICB therapy is administered intravenously. The ICB therapy may comprise one or more inhibitors of CTLA-4, PD-1, PD-L1, PD-L2, LAG-3, BTLA, B7H3, B7H4, TIM3, KIR, or A2aR. For example, the ICB therapy comprises an anti-PD1 antibody and/or an anti-CTLA4 antibody. In some aspects, the anti-PD1 antibody is nivolumab, pembrolizumab, pidillizumab, AMP-514, REGN2810, CT-011, BMS 936559, MPDL328OA or AMP-224. In particular aspects, the anti-CTLA-4 antibody is tremelimumab or ipilimumab.

In some aspects, the method further comprises administering at least one additional anti-cancer therapy. In certain aspects, the anti-cancer therapy is chemotherapy, immunotherapy, targeted therapy, surgery, radiotherapy, or biotherapy. In some aspects, the anti-cancer therapy is administered orally, intravenously, intraperitoneally, intratracheally, intratumorally, intramuscularly, endoscopically, intralesionally, percutaneously, subcutaneously, topically, regionally, or by direct injection or perfusion. In some aspects, the GPER1 agonist and/or at one additional anti-cancer therapy is administered simultaneously. In certain aspects, the GPER1 agonist is administered prior to the at least one additional anti-cancer therapy.

In another embodiment, there is provided a composition comprising an effective amount of GPER1 agonist and/or Myc inhibitor for use in the treatment of BRAF mutant cancer in a subject.

In some aspects, the subject is male. In particular aspects, the BRAF mutation is V600E. In certain aspects, the subject has undergone surgical resection. In some aspects, the cancer is melanoma, such as high-risk resectable melanoma or metastatic melanoma. In some aspects, the BRAF and MEK-targeted therapy is dabrafenib and trametinib. The dafrafenib may be administered at a dose of 100-200 mg, such as 150 mg and trametinib may be administered at a dose of 1-5 mg, such as 2 mg.

In some aspects, the GPER agonist is G-1, fulvestrant, raloxifene, or tamoxifen. The GPER1 agonist may be administered as an adjuvant therapy. In certain aspects, the inhibitor of MYC is a small molecule antagonist, CRABBP inhibitor, BET inhibitor, CDK7 inhibitor, CDK9 inhibitor, mTOR inhibitor, anti-Myc antibody or antigen-binding fragment thereof, an aptamer, siRNA, shRNA, or microRNA. The BET inhibitor may be JQ1 or GSK525762.

In further aspects, the composition further comprises administering an immune checkpoint blockade (ICB) therapy. The ICB therapy may comprise one or more inhibitors of CTLA-4, PD-1, PD-L1, PD-L2, LAG-3, BTLA, B7H3, B7H4, TIM3, KIR, or A2aR. For example, the ICB therapy comprises an anti-PD1 antibody and/or an anti-CTLA4 antibody. In some aspects, the anti-PD1 antibody is nivolumab, pembrolizumab, pidillizumab, AMP-514, REGN2810, CT-011, BMS 936559, MPDL328OA or AMP-224. In particular aspects, the anti-CTLA-4 antibody is tremelimumab or ipilimumab.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

The following drawings form part of the present specification and are included to further demonstrate certain aspects of the present disclosure. The present disclosure may be better understood by reference to one or more of these drawings in combination with the detailed description of specific embodiments presented herein.

FIGS. 1A-1C: Patient cohort neoadjuvant BRAF^{V600mut}-targeted therapy: A) Schema B) Pathological responses and C) Menopausal status.

FIG. 2: Transcriptomic analysis of tumor samples from patients of the initial clinical trial cohort that underwent gene expression profiling with RNA sequencing.

FIG. 3: Pathway-level enrichment of MYC signaling and WNT-beta-catenin signaling in pCR tumors.

FIGS. 4A-4B: Analysis of sex-chromosome-originating genes within the differentially expressed gene set at baseline (A) and greater representation of down-regulated X chromosome genes in on-treatment samples (B).

FIG. 5: Differentially expressed genes involved in estrogen responses demonstrating relatively more enrichment of estrogen response genes in the pre-treatment tumors of pCR patients compared with non-responders.

FIGS. 6A-G: (A-G) Sex dimorphism in response to neoadjuvant targeted therapy further using the Braf^{V600E}/Pten^{-/-} (BP) syngeneic murine melanoma model (female genotype) in C57Bl/6 mice.

FIGS. 7A-7D: Sex dimorphism in response to neoadjuvant targeted therapy further using the Braf^{V600E}/Pten^{-/-} (BP) syngeneic murine melanoma model (female genotype) in nude mice A) males and females B) females C-D) males.

FIG. 8: Comparative male and female melanoma cell expression of GPER1 (top) and cMYC (bottom) at baseline and after the indicated durations of net IC50-dosing of dabrafenib plus trametinib demonstrating sex-dimorphic expression.

### DESCRIPTION OF ILLUSTRATIVE EMBODIMENTS

Significant progress has been made in the treatment of BRAF-mutant melanoma with the use of targeted therapy (TT), and these agents are now being used in earlier stage disease. The results of a phase II trial were recently reported investigating neoadjuvant TT in patients with high-risk resectable melanoma, showing high RECIST and pathologic complete response (pCR) rates (85% and 58%, respectively). Importantly, pCR was associated with a high likelihood of long-term benefit. Interestingly, in this cohort it was noted that a significant proportion of patients achieving a pCR were female (5/7, 71%). This trend was maintained in a retrospective cohort of patients receiving similar TT off-protocol (pCR at surgery in 8/21 patients, all female), and continues in ongoing accruals per-protocol to date (pCR at surgery in 4/9 patients, all female).

To explore the mechanism by which gender might be influencing therapeutic response, transcriptomic data from available pre-treatment tumors was examined and estrogen signaling pathways were found to be enriched in CR patients. A panel of patient-derived BRAF^{V600mut} melanoma cell lines (n=3 male, n=3 female) were analyzed and a markedly dimorphic expression of G Protein-Coupled Estrogen Receptor 1 (GPER1) was identified, being virtually absent in male melanoma cells. MYC signaling represented a potential downstream pathway utilized preferentially in female relative to male melanoma cells. Using a syngeneic Braf^{V600E}/Pten^{-/-} murine melanoma model, enhanced tumor control was demonstrated by combination TT in female animals, consistent with the clinical observation. Together, these data suggest an underlying interaction between a sex-specific environmental factor and sex-based differences in signal pathway utilization in melanoma cells. The present studies found that pharmacologic interventions implicate the androgen-androgen receptor axis and the intratumoral estrogen-androgen balance in this phenomenon, raising the possibility for hormonal manipulation to augment targeted therapy responses in patients.

Accordingly, in certain embodiments, the present disclosure provides methods for targeting sex hormones and androgen receptor signaling for the treatment of cancer, such as to enhance BRAF and RAS targeting in melanoma and other cancers. In certain aspects, methods are provided for treating cancer by administering an androgen receptor modulator, such as an antagonist, in combination with a BRAF and MEK-targeted therapy.

Numerous studies demonstrating estrogen pregenomic signaling in GPER-1-positive, ER-negative cells indicate that GPER-1 can act as a “stand alone” receptor. In addition to the fact that ER and GPER-1 are linked to different signaling mechanisms in reproductive cancers, their actions are independent by several measures, including the following facts: 1) independent expression of ER and GPER-1 is observed in breast tumors and in cultured breast cancer cells lines; 2) ER and GPER-1 display different binding affinities for various estrogens, phytoestrogens, and xenoestrogens and are differentially activated by them; 3) ER antagonists serve as GPER-1 agonists; 4) distinct functions of GPER have been identified using selective GPER-1 agonists and antagonists; 5) ER and GPER null mice exhibit distinct phenotypes (9-11); and finally 6) GPER-1 and ER differentially associate with markers of female reproductive cancers.

Further, in certain embodiments, the present disclosure provides methods of targeting the mechanisms underlying this phenomenon, including estrogen, GPER1, and MYC, to enhance therapeutic responses and outcomes in patients. For example, a GPER1 agonist, such as G-1, and/or a MYC inhibitor may be administered in combination with BRAF and MEK-targeted therapy to treat BRAF-mutant cancer, such as melanoma. The targeted therapy may comprise any drug that inhibits MYC transcription, or induces degradation, or targets antagonists of GPER1.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The terms “about”, “substantially” and “approximately” mean, in general, the stated value plus or minus 5%.

As used herein, the terms “treat”, “treatment”, “treating”, and the like refer to the process of ameliorating, lessening, or otherwise mitigating the symptoms of a disease or condition in a subject by, for example, administering a therapeutic agent to the subject, or by performing a surgical, clinical, or other medical procedure on the subject.

As used herein, the terms “subject” or “patient” are used interchangeably herein to refer to an individual, e.g., a human or a non-human organism, such as a primate, a mammal, or a vertebrate.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to affect such treatment or prevention of the disease.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

### II. METHODS OF USE

In some embodiments, the present disclosure provides methods for treating or delaying progression of cancer comprising administering an androgen receptor modulator, GPER1, estrogen, or MYC targeted therapy. The subject may have BRAF mutated cancer, such as melanoma. The subject may be a male. The subject may be determined to have a low expression of estrogen, GPER1, and/or MYC. The subject may be further administered a BRAF-targeted therapy and/or immune checkpoint blockade.

Targeted therapies may comprise GPER1 agonists, such as G-1, fulvestrant, raloxifene, and tamoxifen. Targeted therapies may comprise Myc targeting agents, such as CRABBP inhibitors and BET inhibitors, such as JQ1 and GSK525762. Other Myc targeting agents may be inhibitors of CDK7, CDK9, mTOR, anti-Myc antibody or antigen-binding fragment thereof; an aptamer, siRNA, shRNA, microRNA, or small molecule inhibitor of Myc protein or a gene encoding the Myc protein; a pharmaceutically acceptable salt thereof; or a combination thereof.

Selective androgen receptor modulators are compounds that bind to androgen receptors to modulate the level of activation of the androgen receptor whilst displacing the binding of endogenous androgens. In some aspects, the androgen receptor modulator is an antagonist. An androgen receptor antagonist (AR antagonist) is a compound that blocks androgen receptor (AR) signaling. Androgen receptor antagonists prevent androgens from expressing their biological effects on responsive tissues. Such compounds may alter the androgen pathway by blocking the respective receptors, competing for binding sites on the receptor, affecting nuclear translocation, DNA binding of the receptor, or affecting androgen production. In the context of the present invention the androgen receptor antagonist can be an anti-androgen, an androgen synthesis inhibitor, a 17 a-hydroxylase/C17,20 lyase (CYP17A1) inhibitor, a 5-alpha-reductase inhibitor, a corticosteroid, a luteinizing hormonereleasing hormone (LH-RH) agonist, or an estrogen agonist. In another embodiment, the androgen receptor antagonist is flutamide, nilutamide, enzalutamide, bicalutamide, ketonazole, abiraterone, abiraterone acetate, orteronel, finasteride, dutasteride, bexlosteride, izonsteride, turosteride, episteride, dexamethasone, prednisone, leuprolide, goserelin, triptorelin, histrelin, or estrogen. In certain aspects, androgen receptor modulation comprises castration. In other aspects, androgen receptor modulation comprises an androgen receptor agonist, such as testosterone.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

A. Combination Therapies

In certain embodiments, the methods provided herein further comprise a step of administering at least one additional therapeutic agent to the subject. All additional therapeutic agents disclosed herein will be administered to a subject according to good clinical practice for each specific composition or therapy, taking into account any potential toxicity, likely side effects, and any other relevant factors.

In certain embodiments, the additional therapy may be immunotherapy, radiation therapy, surgery (e.g., surgical resection of a tumor), chemotherapy, bone marrow transplantation, or a combination of the foregoing. The additional therapy may be targeted therapy. In certain embodiments, the additional therapy is administered before the primary treatment (i.e., as adjuvant therapy). In certain embodiments, the additional therapy is administered after the primary treatment (i.e., as neoadjuvant therapy.

In certain embodiments, the additional therapy comprises an immunotherapy. In certain embodiments, the immunotherapy comprises an immune checkpoint inhibitor.

A targeted therapy may be administered before, during, after, or in various combinations relative to an additional cancer therapy. The administrations may be in intervals ranging from concurrently to minutes to days to weeks. In embodiments where the targeted therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the targeted therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below targeted therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above,

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation, and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates (ADCs) comprise monoclonal antibodies (MAbs) that are covalently linked to cell-killing drugs and may be used in combination therapies. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. Exemplary ADC drugs inlcude ADCETRIS® (brentuximab vedotin) and KADCYLA® (trastuzumab emtansine or T-DM1).

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, erb b2 and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies include immune adjuvants, e.g., Mycobacterium bovis, Plasmodium falciparum, dinitrochlorobenzene, and aromatic compounds); cytokine therapy, e.g., interferons α, β, and γ, IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies. Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody that may be used. Pembrolizumab, also known as MK-3475, Merck 3475, lambrolizumab, KEYTRUDA®, and SCH-900475, is an exemplary anti-PD-1 antibody. CT-011, also known as hBAT or hBAT-1, is also an anti-PD-1 antibody. AMP-224, also known as B7-DCIg, is a PDL2-Fc fusion soluble receptor.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. An exemplary anti-CTLA-4 antibody is ipilimumab (also known as 10D1, MDX-010, MDX-101, and Yervoy®) or antigen binding fragments and variants thereof. In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin.

B. Pharmaceutical Compositions

In another aspect, provided herein are pharmaceutical compositions and formulations comprising a ICB therapy and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of aqueous solutions, such as normal saline (e.g., 0.9%) and human serum albumin (e.g., 10%). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zinc-protein complexes); and/or non-ionic surfactants such as polyethylene glycol (PEG).

### III. KITS

In some embodiments, a kit that can include, for example, one or more media and components for the detection of panel of predictive markers is provided. Such components may comprise reagents, such as primers or antibodies, for the detection of panel of markers in tumor samples that might include GPER1, MYC, ERK, and/or estrogen to predict the response to immune-oncology therapies. The reagent system may be packaged either in aqueous media or in lyophilized form, where appropriate. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The components of the kit may be provided as dried powder(s). When reagents and/or components are provided as a dry powder, the powder can be reconstituted by the addition of a suitable solvent. It is envisioned that the solvent may also be provided in another container means. The kits also will typically include a means for containing the kit component(s) in close confinement for commercial sale. Such containers may include injection or blow molded plastic containers into which the desired vials are retained. The kit can also include instructions for use, such as in printed or electronic format, such as digital format.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Targeting Sex Hormones and Androgen Receptor Signaling to Enhance Responses to BRAF and RAS Targeting in Melanoma and Other Cancers

The standard-of-care management of patients with resectable stage III melanoma or resectable oligometastatic melanoma is upfront surgical resection, followed by adjuvant therapy. The long-time standard adjuvant systemic agent was interferon, which has proven relapse-free survival benefit, questionable overall survival benefit, and high attendant toxicity rates leading to premature discontinuation in a high proportion of patients. In recent years, combination targeted therapy (dabrafenib plus trametinib) and, more recently, anti-PD-1 based therapy (nivolumab) have received FDA approval for adjuvant use in this disease, however mature long-term survival data are awaited. A randomized phase II clinical study was previously conducted to determine event-free survival outcomes between patients offered standard-of-care management for resectable stage III or oligometastatic melanoma versus neoadjuvant plus adjuvant targeted therapy with dabrafenib and trametinib. Neoadjuvant targeted therapy for 8 weeks was associated with a very high radiographic response rate (85%) and striking pathologic complete response rate (pCR; 58%) as determined from microscopic evaluation of surgical resection specimens.

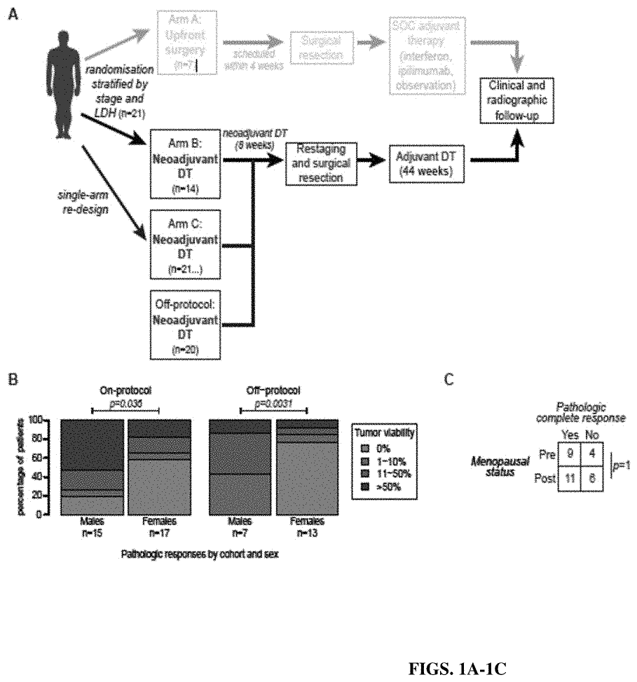

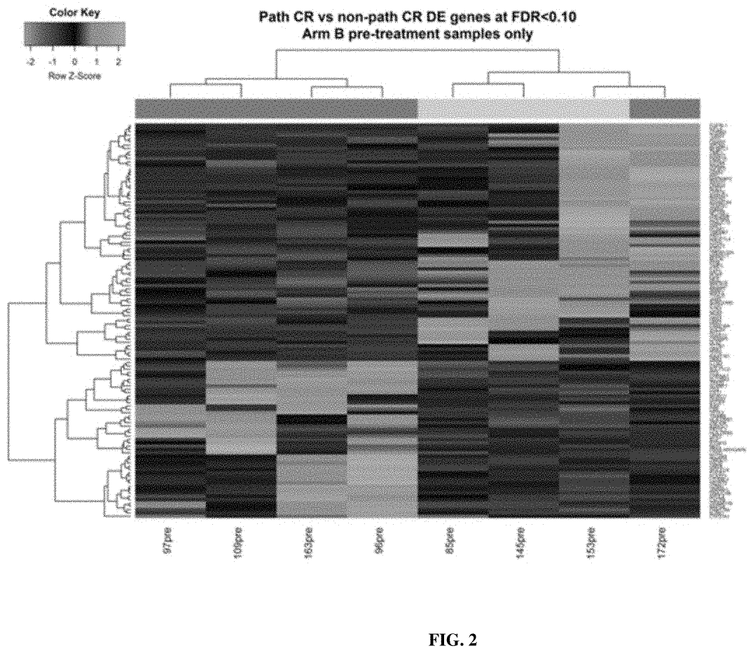

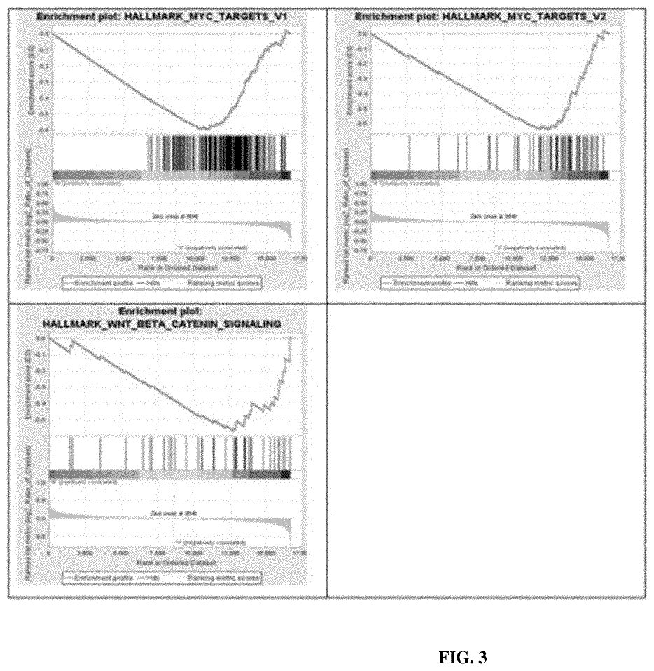

Clinical outcomes stratified by sex were evaluated in the original (n=12 evaluable) cohort of patients treated with neoadjuvant dabrafenib plus trametinib (neoDT) on the COMBI-NEO trial (NCT02231775) (Pinto et al., 2018), and a statistically-significant over-representation of pathologic complete responders (pCR) was found in females versus males (5/5 females, 2/7 males, p=0.028). Following early closure of the trial due to clear benefit in the experimental arm and re-design as a single-arm study, an additional 21 patients were recruited and evaluable for pathologic response following 8 weeks of neoDT. Across this expanded cohort, pathologic complete responses were observed in 13 patients; 10/17 females and 3/15 males (p=0.036). Additionally, a retrospective cohort of patients were identified to be treated with analogous neoadjuvant BRAF^{V600mut}-targeted therapy off-protocol largely due to logistical reasons (n=2 dabrafenib, n=1 encorafenib plus binimetinib, n=18 dabrafenib plus trametinib), and a pCR rate of 50% was observed with a marked sex bias favoring females (10/13 females, 0/7 males, p=0.0031). (FIG. 1). Within the female subset, there was no association between menopausal status (pre- vs post-) and pCR (pCR n=9 pre-menopausal, n=11 post-menopausal; non-pCR n=4 pre-menopausal, n=6 post-menopausal; p=1) (FIG. 1C).

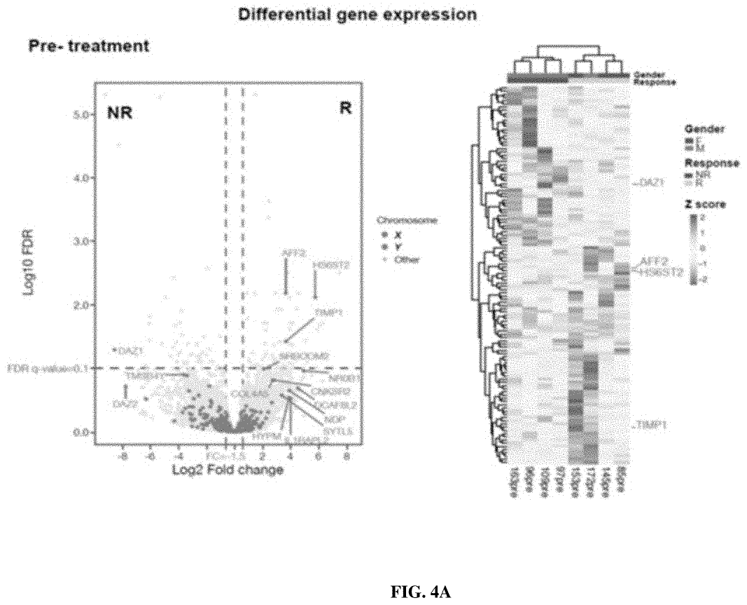

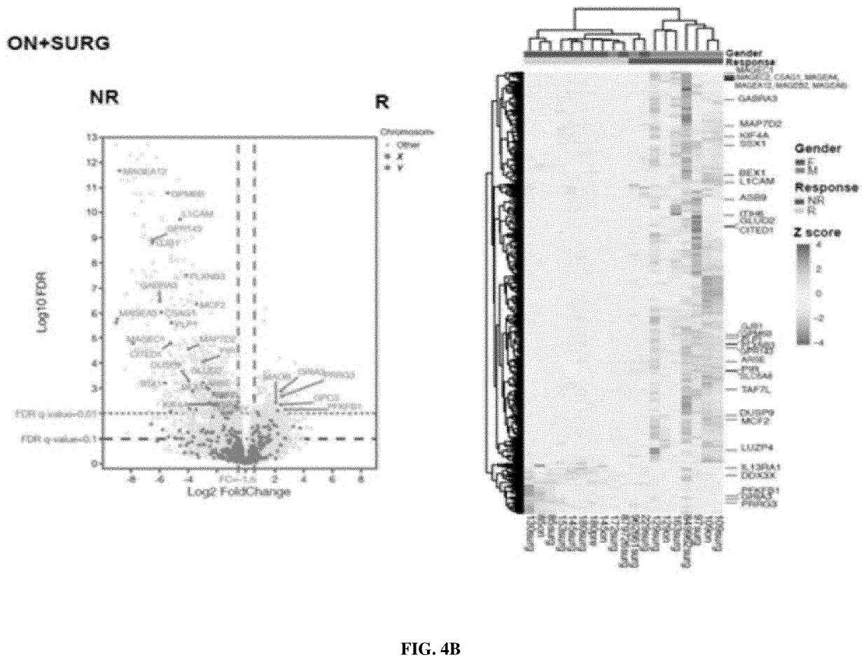

To explore molecular mechanisms underlying the observed sex-difference in response to neoadjuvant therapy, transcriptomic data was analyzed from patient tumor samples of the initial clinical trial cohort that underwent gene expression profiling with RNA sequencing. In pre-treatment samples, tumors from patients who subsequently achieved pCR to therapy demonstrated upregulation of several genes involved in mitogenic or cell-survival pathways, including IGF-1, MAPK and WNT-signaling (FIG. 2), with gene set enrichment analysis revealing pathway-level enrichment of MYC signaling and WNT-beta-catenin signaling in pCR tumors (FIG. 3). Specific analysis of sex-chromosome-originating genes within the differentially-expressed gene set revealed minimal significant differences at baseline (FIG. 4A) and greater representation of down-regulated X chromosome genes in on-treatment samples (FIG. 4B), likely reflecting treatment-related differential tumor content between male and female patients after treatment initiation.

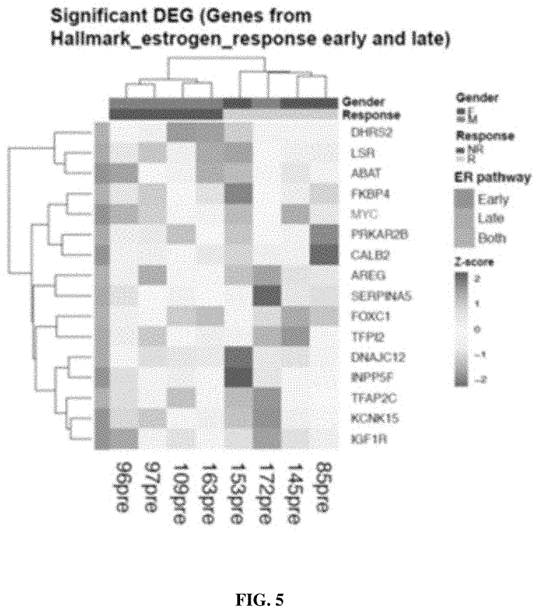

Additionally, based on the sex-based differences observed, differentially-expressed genes involved in estrogen responses were evaluated by querying the annotated “Hallmark estrogen response early and late” gene set, demonstrating relatively more enrichment of estrogen response genes in the pre-treatment tumors of pCR patients compared with non-responders, consistent with these tumors being more inherently estrogen-responsive (FIG. 5). Notably, these genes included MYC, which was previously identified as a key pathway-level enrichment within pCR patient tumors (FIG. 3).

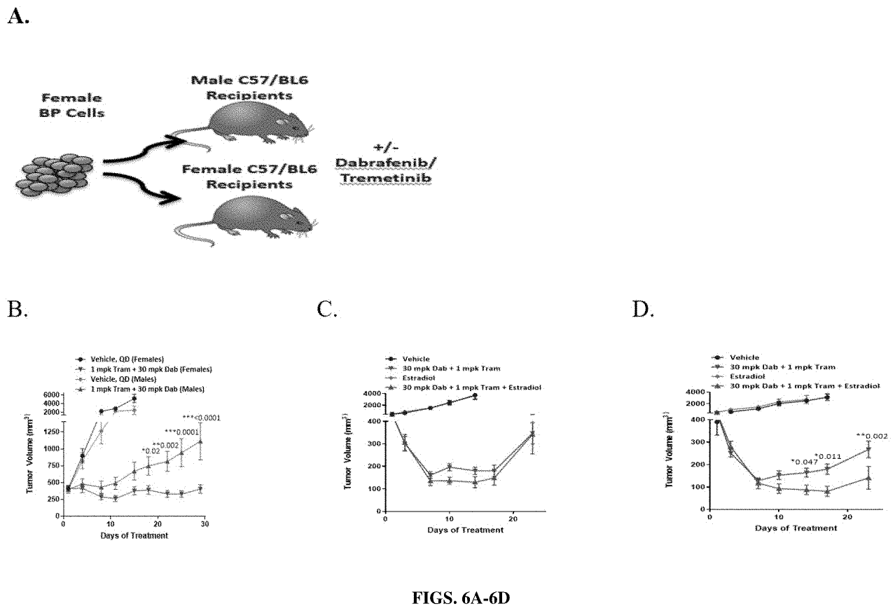

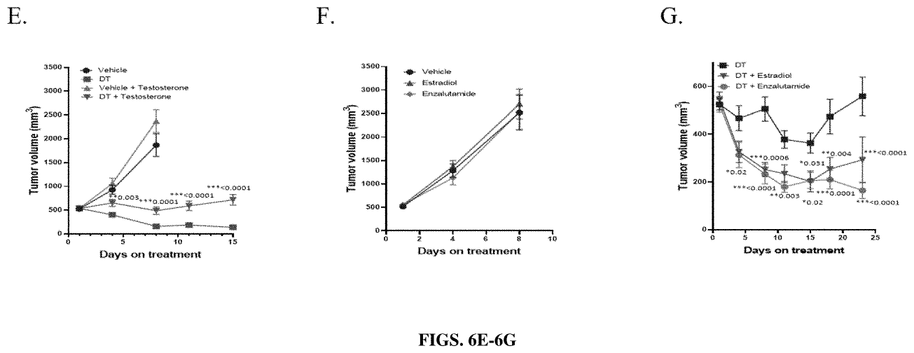

It was sought to evaluate sex dimorphism in response to neoadjuvant targeted therapy further using the Braf^{V600E}/Pten^{-/-} (BP) syngeneic murine melanoma model (female genotype) in C57Bl/6 mice. Following subcutaneous tumor inoculation and once tumors became established, treatment with dabrafenib plus trametinib (DT) resulted in stability or slight regression of tumors in female animals, whilst tumors continued to grow in male animals. Given indications of a hormonal basis from patient data, we next evaluated the effect of administration of exogenous estradiol, testosterone, or androgen receptor blockade with enzalutamide on tumor control by DT (FIG. 6).

In parallel, hormonal perturbations were evaluated in vitro using a panel of BRAFV600E melanoma cell lines derived from melanoma patients of the institution. In cell culture, no significant effects of estradiol, testosterone, or enzalutamide were found on the proliferative, cell cycle, or apoptosis response to DT treatment, and minimal expression of the androgen receptor in melanoma cells by Western blot. Together these data indicate a probable contribution of microenvironmental/stromal cellular components.

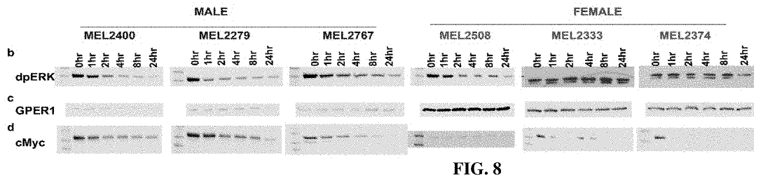

Given the transcriptomic implication of MYC signaling and estrogen responsiveness in development of a pCR response to targeted therapy, the expression of estrogen receptors was explored including the non-canonical G-protein coupled estrogen receptor GPER1, and cMYC, in model human melanoma cell lines derived from patient tumors harvested under the institutional tumor infiltrating lymphocyte (TIL) program. Strikingly, a clear sex dimorphism was observed in expression of GPER1 between male and female cell lines (virtually unexpressed in male cells), associated with a higher level of cMYC in male cells (FIG. 8).

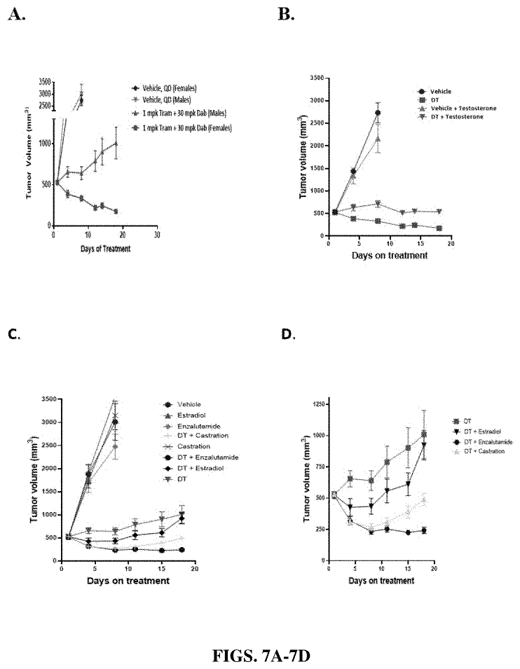

Sex differences in immunity are well described in the literature (Klein and Flanagan, 2016), and may be relevant to the observed sex bias in response to DT considering the known early on-treatment immunogenic effects of MAPK blockade in melanoma (Boni et al., 2010). To evaluate the relevance of an immune contribution to the enhanced responses to DT observed in female mice, tumor growth curves were evaluated using the BP model implanted into immunodeficient mice. Notably, data indicated that AR blockade is more effective than estrogen supplementation. This critical observation suggests the key role of androgen in MAPK blockade in melanoma (FIG. 7). Thus, targeting sex hormones and androgen receptor signaling can be used to enhance BRAF and RAS targeting in melanoma and other cancers.

TABLE 1
Summary of clinical characteristics of patients enrolled in Neo-
adjuvant targeted therapy trial (on and off-protocol study)

| | | On-protocol | | | On and Off-protocol | | |
|---|---|---|---|---|---|---|---|
| Variable | | F (n = 17) | M (n = 18) | P | F (30) | M (25) | P |
| Age (median, range) | | 53 (31, 79) | 58 (23, 88) | 0.8 | 53 (31, 79) | 60 (20, 88) | 0.4 |
| BMI (median, range) | | 28 (22, 40) | 30 (21, 42) | 0.5 | 28 (21, 44) | 30 (21, 42) | 0.4 |
| Stage | 3b | 8 | 3 | 0.06 | 9 | 4 | 0.2 |
| | 3c | 9 | 13 | | 17 | 19 | |
| | 3d | 0 | 0 | | 3 | 0 | |
| | 4 | 0 | 2 | | 1 | 2 | |
| BRAFV600 | E | 15 | 13 | 0.05 | 27 | 19 | 0.04 |
| | K | 0 | 5 | | 1 | 6 | |
| ECOG | No | 16 | 14 | 0.6 | 28 | 18 | 0.1 |
| | Yes | 1 | 3 | | 2 | 6 | |
| LDH | No | 14 | 16 | 0.6 | 22 | 22 | 0.2 |
| elevated | Yes | 3 | 1 | | 8 | 2 | |
| pCR | No | 7 | 12 | 0.04 | 10 | 19 | 0.0002 |
| | yes | 10 | 3 | | 20 | 3 | |
| Metastatic | Denovo | 7 | 7 | 1 | 11 | 11 | 0.6 |
| disease | Recurrent | 10 | 11 | | 19 | 14 | |

F—Female, M—Male, Staging is based on AJCC staging system 8th Edition. P values were calculated using Fisher's exact test for categorical variables and Mann-Whitney/Wilcoxon test for continuous variable.

### Example 2—Materials and Methods

Clinical Cohorts:

Patients enrolled in the initial clinical trial cohort were as previously described (Amaria et al., 2018). Briefly, patients ≥18 years old with histologically-proven clinical stage III or oligometastatic stage IV BRAFV600E/K melanoma deemed resectable by multidisciplinary consensus and measurable disease by RECIST 1.1 criteria were enrolled and those randomized to the experimental arm received 8 weeks of neoadjuvant dabrafenib (150 mg PO BD) plus trametinib (2 mg PO daily) prior to surgical resection, followed by up to 44 weeks of adjuvant dabrafenib and trametinib. Radiographic responses to neoadjuvant therapy were determined at week 8 prior to surgery and pathologic responses were determined by microscopic examination of the complete surgical specimen by a melanoma pathologist, including SOX10 immunostains when applicable to confirm presence or absence of viable melanoma cells.

RNA Sequencing:

Tumor biopsies were obtained as feasible by punch or core biopsy prior to and during the neoadjuvant treatment period. Specimens were also obtained from the surgical specimens as a post-neoadjuvant treatment time point. Fresh-frozen tumor biopsy material was used for RNA sequencing library preparation. Total RNA was extracted from snap-frozen tumor specimens using the AllPrep DNA/RNA/miRNA Universal Kit (Qiagen) following assessment of tumor content by a Pathologist, and macrodissection of tumor bed if required. RNA quality was assessed on an Agilent 2100 Bioanalyzer using the Agilent RNA 6000 Nano Chip with smear analysis to determine DV200 and original RNA concentration. Based on RNA quality, 40-80 ng of total RNA from each sample then underwent library preparation using the Illumina TruSeq RNA Access Library Prep kit according to the manufacturer's protocol. Barcoded libraries were pooled to produce final 10-12 plex pools prior to sequencing on an Illumina NextSeq 500 sequencer using one high-output run per pool of 76 bp paired-end reads, generating 8 fastq files (4 lanes, paired reads) per sample.

RNA Sequencing Data Processing:

RNA-seq FASTQ files were first processed through FastQC (v0.11.5) (Andrews et al., 2016), a quality control tool to evaluate the quality of sequencing reads at both the base and read levels. Reads having 15 contiguous low-quality bases (phred score <20) were removed from the FASTQ files prior to STAR 2-pass alignment (v2.5.3) [12] with default parameters to generate one BAM file for each sequencing event. After that, RNA-SeQC (v1.1.8) (DeLuca et al., 2015) was used to generate quality control metrics including read counts, coverage, and correlation. A matrix of Spearman correlation coefficients amongst all sequenced samples was subsequently generated by RNA-SeQC and after careful review the sequencing data generated from one library pool that showed poor correlation with other library pools from the same RNA sample was removed before sample-level merging of BAM files.

HTSeq-count (v0.9.1) (Anders et al., 2016) tool was applied to aligned RNA-seq BAM files to count how many aligned reads overlapped with the exons of each gene. The raw read counts generated from HTSeq-count were normalized into fragments per kilobase of transcript per million mapped reads (FPKM) using the RNA-seq quantification approach suggested by the bioinformatics team of NCI Genomic Data Commons (GDC). Briefly, FPKM normalizes read count by dividing it by the gene length and the total number of reads mapped to protein-coding genes using a calculation described below:

$$FPKM = \frac{{RC}_{g} * {10}^{9}}{{RC}_{pc} * L}$$

RC_{g}, number of reads mapped to the gene; RC_{pc}: number of reads mapped to all protein-coding genes; L, length of the gene in base pairs (calculated as the sum of all exons in a gene). The FPKM values were then log 2-transformed for further downstream analyses.

RNA Sequencing Differential Expression and Gene Set Enrichment Analysis:

The HTSeq normalized read count data for all expressed coding transcripts was processed by DESeq2 (v3.6) (Love et al., 2014) software to identify differentially expressed genes (DEGs) between two response (responder vs. non-responder) groups. A cut-off of gene expression fold change of ≥2 or ≤−0.5 and a FDR q-value of <0.05 was applied to select the most differentially expressed genes. Gene set enrichment analyses were performed using the GSEA software developed at the Broad Institute, using vst-normalised (Huber et al., 2002) input transcriptome expression data and querying the MSigDB Hallmark gene sets with default parameters.

Cell Line, Reagents and Drugs:

In vivo grade Trametinib, Dabrafenib, and Enzulamide were purchased from Chemieteck (Indianapolis, Ind.). Both Trametinib and Dabrafenib were formulated in 0.5% Hydroxypropylmethylcellulose (cat # H8384-100G, Sigma) and 0.2% Tween 80 (Cat #, BDH7781-2, VWR) in distilled water (pH 8.0) separately and then mixed (1:1) before dosing. Enzulamide was formulated in 1% carboxymethyl cellulose (Sigma), 0.1% Tween-80, 5% DMSO. Both controlled release 17β-estradiol (cat # SE-121) and Testosterone (cat # SA-151) pellets for 60 days were purchased from Innovative Research of America (Sarasota, Fla.).

Animals and Xenograft Model:

Female or Male C57BL/6 mice (strain code: 0000664, purchased from Jackson Lab), aged 6 to 12 weeks and weighing approximately 20 to 25 g were used for in vivo studies. Female or male CD-1 nude mice (strain code: 086, purchased from Charles River Laboratories), aged 6 to 12 weeks and weighing approximately 25-40 g were used for the immunodeficient model in vivo study. Animal health was monitored daily by observation and sentinel blood sample analysis. Animal experiments were conducted in accordance with the Guideline of IACUC at MDACC.

BP cells were scaled up in DMEM culture media supplemented with 10% FBS, harvested, and prepared so that each mouse received 0.8×10⁶ cells in 0.2 mL PBS. nCells were implanted subcutaneously in the right flank of each mouse. For some male or female mice, castration or oophorectomy were required and performed two weeks before treatment or before cell implantation. 17β-estradiol and Testosterone pellets were implanted subcutaneously in the left flank one week before treatment. Trametinib at 1 mg/kg and Dabrafenib at 30 mg/kg were suspended at concentrations as needed in an aqueous vehicle containing 0.5% Hydroxypropylmethylcellulose and 0.2% Tween 80 in distilled water and adjusted to pH 8.80 with diluted NaOH solution. Enzulamide at 10 mg/kg was suspended in 1% carboxymethyl cellulose (Sigma), 0.1% Tween-80, 5% DMSO.

BP tumors were monitored by caliper before randomly sorting and dividing into experimental groups (n=10 mice per group). Treatment was started from day 14 to 17 days post-implantation, depending on mouse strain and mouse genders. Vehicle controls, mixture of Trametinib at 1 mg/kg and Dabrafenib at 30 mg/kg, or Enzulamide at 10 mg/kg were given orally using a sterile 1-mL syringe and 18-gavage needle for 21 days. Dosing was 5 hours apart between administration of Trametinib+Dabrafenib and Enzulamide for these specific treatment groups.

Tumor volume was calculated using the following formula: [L×(W²)/2] (in which L+length of tumors; W=width of tumor). Tumor and plasma were harvested 4 hours after the last dose. Tumors were snap of frozen and the plasma were divided for monitoring drug concentrations and hormone levels.

Melanoma Cell Lines:

Melanoma cell lines derived from tumors of patients treated at UT MD Anderson Cancer Center under the TIL program were obtained from Dr. Chantale Bernatchez and Dr. Scott Woodman. For these studies, n=5 male (2373, 2400, 2650, 2800 and 624-mel) and n=5 female (2153, 2333, 2379, 2508, 2510) lines were selected based on carriage of BRAF^{V600E/K} mutations and avoidance of prior exposure in vivo to MAPK blockade therapies. Cells were maintained in phenol red-free RPMI-1640 supplemented with 10% heat-inactivated FBS, 1% GlutaMAX and 1% Pencillin/Streptomycin (all Gibco). LNCaP, MCF7 and PC3 cell lines were obtained from institutional stocks and maintained in RPMI-1640 supplemented as above (LNCaP, MCF7) or DMEM/F12 supplemented as above (PC3). All cell lines were confirmed mycoplasma negative using the Lonza MycoALERT kit prior to use, and authenticated by STR profiling via the MD Anderson Characterized Cell Line Core.

Chemicals:

For hormonal co-treatments, cells were treated at fixed clinically-relevant concentrations of dabrafenib 1 μM (S2807, Selleck Chemicals) and trametinib 100 nM (S2673, Selleck Chemicals); testosterone 100 nM, 500 nM or luM (T1500, Sigma-Aldrich); estradiol 100 nM (E2758, Sigma-Aldrich); or enzalutamide 1 μM (S1250, Selleck Chemicals).

Proliferation Assays:

Combination dabrafenib plus trametinib IC50s were determined empirically from 72-hour treatment MTS proliferation assays as previously described [27852308] based on dabrafenib monotherapy (5-fold serial dilutions covering dose range 0-5000 nM), trametinib monotherapy (4-fold serial dilutions covering dose range 0-4096 pM), and subsequently the same trametinib dose range at constant ˜IC70 dabrafenib dose curves. Combination net-IC50 doses were then used to treat each cell line for t=0, 1, 2, 4, 8.24 hours prior to protein harvest. CyQUANT Cell Proliferation Assay Kit (C7026, Thermofisher).

Apoptosis Assays:

Cells were plated into 6-well plates at 250,000 cells/well in full growth media and allowed to adhere overnight prior to drug treatment as shown in results in reduced-serum medium (5% FBS, phenol red-free RPMI-1640). At +72 hours, cells were harvested by gentle trypsinisation and collected into ice-cold PBS, followed by staining using the Vybrant Apoptosis Assay Kit #4, YO-PRO-1/PI (V13243, Thermofisher) as per the manufacturer's instructions. Data acquisition by flow cytometry was performed within 2 hours of cell harvest and analyzed in FlowJo after gating on cell size, singlets, and applying Curly Quadrant gates to PI vs YO-PRO-1 fluorescence.

Western Blots:

Protein was extracted from treated melanoma cells pelleted at 4° C. using Pierce RIPA lysis buffer containing protease and phosphatase inhibitors (cOmplete mini, PhosSTOP) as per manufacturer's recommendations. Protein concentration was determined by the BCA assay with reference to a bovine serum albumin standard (ThermoFisher) and samples adjusted to uniform concentrations prior to addition of Laemmli sample buffer (4×) and reducing agent (DTT). Samples underwent heat denaturation (70° C., 5 min) and 20 μg was immediately loaded onto 4-12% SDS-polyacrylamide gels for sample separation by electrophoresis followed by transfer to nitrocellulose membranes. Membranes were blocked for 1 hour at room temperature in Odyssey Blocking Buffer (LI-COR) and then stained with 1:2000 rabbit anti-Androgen receptor [D6F11] XP rabbit mAb (#5153, Cell Signaling Technologies), 1:10,000 rabbit anti-GAPDH [14C10] mAb (#2118, Cell Signaling Technologies) overnight at 4° C. prior to secondary antibody staining with 1:40,000 IRDye 680RD goat anti-rabbit IgG secondary antibody (#926-68071, LI-COR) for 1 hour at room temperature and visualization on the LI-COR Odyssey system. Membranes were stained for either diphospho-ERK1/2, anti-human GPER1 (1:200 overnight), pRb Ser807/811, or cMYC (1:100 overnight) prior to secondary antibody staining (1 hr) and visualization on the LICOR Odyssey system.

Immunofluorescence:

Unstained sections cut from FFPE-preserved tumor biopsies of patients in the initial trial cohort were stained with DAPI, anti-human GPER1 and anti-human cMYC.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Amaria, R. N., et al., Neoadjuvant plus adjuvant dabrafenib and trametinib versus standard of care in patients with highrisk, surgically resectable melanoma: a single-centre, open-label, randomised, phase 2 trial. Lancet Oncol, 2018. 19(2): p. 181-193.
Anders, S., P. T. Pyl, and W. Huber, HTSeq—a Python framework to work with high-throughput sequencing data. Bioinformatics, 2015. 31(2): p. 166-9.
Andrews, M. C., et al., Systems analysis identifies miR-29b regulation of invasiveness in melanoma. Mol Cancer, 2016.15(1): p. 72.
Boni, A., et al., Selective BRAFV600E inhibition enhances T-cell recognition of melanoma without affecting lymphocyte function. Cancer Res, 2010. 70(13): p. 5213-9.
DeLuca, D. S., et al., RNA-SeQC: RNA-seq metrics for quality control and process optimization. Bioinformatics, 2012.28(11): p. 1530-2.
Dobin, A., et al., STAR: ultrafast universal RNA-seq aligner. Bioinformatics, 2013. 29(1): p. 15-21.
Huber, W., et al., Variance stabilization applied to microarray data calibration and to the quantification of differential expression. Bioinformatics, 2002. 18 Suppl 1: p. S96-104.
Klein, S. L. and K. L. Flanagan, Sex differences in immune responses. Nat Rev Immunol, 2016. 16(10): p. 626-38.
Love, M. I., W. Huber, and S. Anders, Moderated estimation of fold change and dispersion for RNA-seq data with DESeq2. Genome Biol, 2014. 15(12): p. 550.
Pinto, J. A., et al., Gender and outcomes in non-small cell lung cancer: an old prognostic variable comes back for targeted therapy and immunotherapy? ESMO Open, 2018. 3(3): p. e000344.

1. A method of treating a BRAF-mutant cancer in a subject comprising administering an androgen receptor (AR) antagonist to the subject.

2. The method of claim 1, wherein the subject is male.

3. The method of claim 1, wherein the BRAF mutation is V600E.

4. The method of claim 1, wherein the AR antagonist is enzalutamide, bicalutamide, lutamide, nilutamide, ketonazole, abiraterone, abiraterone acetate, orteronel, finasteride, dutasteride, bexlosteride, izonsteride, turosteride, episteride, dexamethasone, prednisone, leuprolide, goserelin, triptorelin, histrelin, or estrogen.

5. (canceled)

6. The method of claim 1, wherein the subject has undergone surgical resection or castration.

7. (canceled)

8. The method of claim 1, further comprising administering a BRAF and MEK-targeted therapy.

9. The method of claim 8, wherein the BRAF and MEK-targeted therapy comprises dabrafenib and/or trametinib.

10. (canceled)

11. The method of claim 9, wherein dafrafenib is administered at a dose of 100-200 mg.

12. (canceled)

13. The method of claim 9, wherein trametinib is administered at a dose of 1-5 mg.

14. (canceled)

15. The method of claim 1, wherein the AR antagonist is administered as an adjuvant therapy.

16. The method of claim 1, wherein the cancer is melanoma.

17. The method of claim 16, wherein the melanoma is high-risk resectable melanoma or metastatic melanoma.

18. The method of claim 1, further comprising administering an immune checkpoint blockade (ICB) therapy.

19. The method of claim 18, wherein the ICB therapy is administered intravenously.

20. The method of claim 18, wherein the ICB therapy comprises one or more inhibitors of CTLA-4, PD-1, PD-L1, PD-L2, LAG-3, BTLA, B7H3, B7H4, TIM3, KIR, or A2aR.

21. The method of claim 18, wherein the ICB therapy comprises an anti-PD1 antibody and/or an anti-CTLA4 antibody.

22. The method of claim 21, wherein the anti-PD1 antibody is nivolumab, pembrolizumab, pidillizumab, AMP-514, REGN2810, CT-011, BMS 936559, MPDL328OA or AMP-224.

23. The method of claim 21, wherein the anti-CTLA-4 antibody is tremelimumab or ipilimumab.

24-28. (canceled)

29. A composition comprising an effective amount of an AR antagonist and a BRAF and MEK-targeted therapy for use in the treatment of BRAF mutant cancer in a subject.

30-34. (canceled)

35. A method of treating a BRAF-mutant cancer in a subject comprising administering a G Protein-Coupled Estrogen Receptor 1 (GPER1) agonist and/or an inhibitor of MYC to the subject.

36-66. (canceled)
